(19) United States
(12) Patent Application Publication
(10) Pub. No.: US 2005/0277948 A1
(43) Pub. Date: Dec. 15, 2005

(54) Apparatus and methods for the administration of a cerclage

(76) Inventors: Leonard Cedars, San Luis Obispo, CA (US); John McMains, San Luis Obispo, CA (US)

Correspondence Address: JONES DAY, 222 EAST 41ST ST, NEW YORK, NY, 10017, US

(21) Appl. No.: 11/148,392

(22) Filed: Jun. 7, 2005

Related U.S. Application Data
(60) Provisional application No. 60/579,567, filed on Jun. 14, 2004.

(51) Int. Cl. A61D 1/10; A61B 17/42; A61B 17/46

(52) U.S. Cl. 606/119

(57) ABSTRACT

An embodiment of the present invention provides a device comprising a primary tube having an interior member slideably mounted within the primary tube. The interior member has a length that is longer than a length of the primary tube so that the interior member protrudes from at least one end of the primary tube. A first cup is fixably mounted on a first end of the primary tube. This first cup has an opening through which the interior member protrudes. A finger hold is fixably mounted on a second end of the primary tube. A second cup is fixably mounted on the end of the interior member that protrudes through the first cup. A handle is fixably mounted on the end of the interior member that protrudes from the second end of the primary tube.

### CROSS REFERENCE TO RELATED APPLICATIONS

This application claims benefit, under 35 U.S.C. § 119(e), of U.S. Provisional Patent Application No. 60/579,567, which is hereby incorporated by reference in its entirety.

### TECHNICAL FIELD OF THE INVENTION

Generally, this invention relates to devices and methods for assisting practitioners who are treating patients with reduced cervical competence. More specifically, the devices and methods of the present invention are intended for patients that present with advanced effacement and dilation of the cervix and the membranes bulging into the vagina.

### BACKGROUND OF THE INVENTION

Cervical incompetence arises when a cervix (the outlet of the uterus) is too weak to stay closed during a pregnancy. It is defined as a condition in which the cervix fails to retain the conceptus during pregnancy. This can result in a preterm birth and possibly the loss of the baby because of the shortened gestational length. Cervical incompetence affects around one percent of pregnant patients and has long been recognized as a potential cause of preterm delivery and recurrent mid trimester abortions. It is believed that cervical incompetence is the cause of 20-25% of all second trimester losses in humans. The cervical incompetence generally shows up in the early part of the second trimester, but possibly as late as the early third trimester in humans.

Women with incompetent cervix typically present with “silent” cervical dilation (i.e., with minimal uterine contractions) between 16 and 28 weeks of gestation. Symptoms include patients that present with significant cervical dilation (2 cm or more) and minimal other symptoms. When the cervix reaches 4 cm or more, active uterine contractions or rupture of membranes may occurs. In fact, short labors with the delivery of an immature fetus or loss of the pregnancy at progressively earlier gestational ages in successive pregnancies is characteristic of reduced competence. Diagnosis of cervical incompetence in humans can be made either manually or with sonography by transvaginal scanning. The diagnosis is made when the cervical os (opening) is greater than 2.5 cm, or the length has shorted to less than 20 mm.

Cervical incompetence can be treated by cervical cerclage sutures. Cervical cerclage involves stitching shut the cervix in order to prevent it from opening before the pregnancy has gone to term. In humans, cervical cerclage can be done preventively at 12 to 14 weeks before the cervix thins out, or as an emergency measure after the cervix has thinned. Cervical cerclage is performed using either general or regional anesthesia. A speculum (an instrument with spoon-like paddles) is inserted into the pregnant woman's vagina to spread the vaginal walls apart for the surgery.

The cerclage procedure can be done in different ways. For instance, the McDonald procedure is done with a 5 mm band of permanent suture that is placed high on the cervix. Such a procedure is used when there is significant effacement of the lower portion of the cervix. It is generally removed at 37 weeks, unless there is a reason to remove it earlier, like infection, preterm labor, premature rupture of the membranes, etc. In the McDonald procedure, as illustrated in FIGS. 8A and 8B, the cervix is closed using four of five bites with a needle to create a purse string around the cervix. In another approach, a special tape can be tied around the cervix and stitched in place. In still another approach, a small incision can be made in the cervix. A special tape is then tied through the cervix to close it. Other cerclage procedures include the Shirodkar operation, the Hefner cerclage (also know as the Wurm procedure), the transabdominal cerclage by Benson and Durfee, and the Lash procedure.

If an incompetent cervix is not diagnosed and treated in earlier stages of pregnancy, the woman's amniotic sac can begin to protrude through her cervix. Moreover, when a woman has preterm labor (PTL) she often experiences premature dilation of the cervix. This allows the fetal (amniotic) membranes to bulge through the cervix into the vagina. No satisfactory device exists in the art to treat the bulging amniotic sac arising during pregnancy as a result conditions such as cervix incompetence or preterm labor. In other words, no satisfactory tool exists to “push” the membranes back through the cervix into the uterine cavity before performing a cerclage to stabilize the cervix.

Some practitioners use the balloon of a Foley Catheter to push the amniotic membranes back through the cervix into the uterine cavity. This is an unconventional use of the Foley Catheter (urinary catheter). Conventionally, the Foley catheter is used to drain urine from the bladder. In such conventional uses, a balloon on the Foley catheter is used to anchor the catheter in place. To treat a bulging amniotic sack, the Foley catheter can be used in an unconventional manner. The Foley catheter balloon is placed inside the cervix and overfilled with at least 50 ml of saline to push the amniotic sac back into the uterine cavity. The cerclage suture can then be placed and tied as the balloon fluid is evacuated. The drawback with this unconventional use of the Foley catheter is that it is not rigid enough to satisfactorily and safely perform the procedure. Moreover, the Foley catheter and related instruments do not provide the visibility and control needed by a practitioner so that the cervix can be sewn up with a cerclage while the amniotic sack is being pushed back into the uterine cavity with the Foley catheter or related instrument.

Given the above background, there is a need in the art for devices that can safely and effectively push amniotic tissue back into the uterine cavity without rupturing the amniotic membranes while at the same time providing the visibility and control needed by the practitioner so that the cervix can be stabilized with a cerclage.

### SUMMARY OF THE INVENTION

The present invention provides an apparatus for making contact with bulging amniotic membranes. The apparatus includes a primary cup and a secondary cup located within an interior of the primary cup. The primary cup is used to make contact with the bulging membranes. Then the secondary cup is elevated to push the membranes back into the uterine cavity. A purse string type cerclage is then placed while the device is in this elevated position. The stitch is pulled tight as the secondary cup is withdrawn from the cervix.

In some embodiments, the membrane elevator is a device approximately 30 cm in length with a 20° bend. There is a primary (outer) and secondary (inner) shaft and “cup”. The cups are at the distal end of the shaft and the secondary cup fits flush inside the primary cup. The primary cup can have a diameter between approximately two cm and eight cm. At the proximal end of the shaft, a thumb ring is used in cooperation with a finger hold. Such a mechanism is similar to a plunger system on a dental syringe. The device is inserted into the vagina up to the cervical os. The primary cup is held firmly against the bulging membrane until the cervix is covered. The secondary, smaller cup is deployed gently pushing the membrane of the amniotic sac upwards and back into the cervix. The cerclage can then be tied and the device removed simultaneously.

An embodiment of the present invention provides a device comprising a primary tube having an interior member slideably mounted within the primary tube. The interior member has a length that is longer than a length of the primary tube so that the interior member protrudes from at least one end of the primary tube. A first cup is fixably mounted on a first end of the primary tube. This first cup has an opening through which the interior member protrudes. A finger hold is fixably mounted on a second end of the primary tube. A second cup is fixably mounted on the end of the interior member that protrudes through the first cup. A handle is fixably mounted on the end of the interior member that protrudes from the second end of the primary tube.

Another embodiment of the present invention provides a method of implementing a cerclage. In the method, a distal end of a device is inserted into the vagina up to the cervical os while the device is in a closed position. The device includes a second cup that is located circumferentially within a first cup of the device. The device is inserted until the first cup is pressing against a uterine wall. Next the second cup is inserted into the cervix of the uterus thereby causing the second cup to be deployed in an extended position that pushes a membrane of the amniotic sac into the cervix. A cerclage of the cervix is initiated. Then, the second cup is withdrawn from the cervix as the cerclage is tightened, thereby implementing the cerclage. In some embodiments, the cerclage is performed in accordance with the McDonald approach. In some embodiments, after step (B) and prior to step (C), the first cup is removed from the vagina.

### BRIEF DESCRIPTION OF THE DRAWINGS

The features and advantages of the present invention will be better understood by reference to the following detailed description, which should be read in conjunction with the accompanying drawings.

FIG. 1 is a perspective view of an embodiment of the membrane elevator in accordance with the present invention.

FIG. 2 is a view of an embodiment of the membrane elevator of the present invention in an extended position.

FIG. 3 is a view of an embodiment of the membrane elevator of the present invention in a closed position.

FIG. 4 is a cross-sectional view of an embodiment of the membrane elevator of the present invention.

FIG. 5 is an end view of an embodiment of the membrane elevator of the present invention illustrating a relationship between a first and second cup.

FIG. 6 is a cross-sectional view of an embodiment of the membrane elevator of the present invention in a closed position.

FIG. 7 is a cross-sectional view of an embodiment of the membrane elevator of the present invention in an extended position.

FIG. 8A illustrates a purse string type cerclage performed using the McDonald procedure in accordance with the prior art.

FIG. 8B provides another embodiment of a cerclage performed in accordance with the prior art.

FIG. 9 is a three dimensional view of the membrane elevator in accordance with another embodiment of the present invention.

Like reference numerals refer to corresponding parts throughout the several views of the drawings.

### DETAILED DESCRIPTION OF THE INVENTION

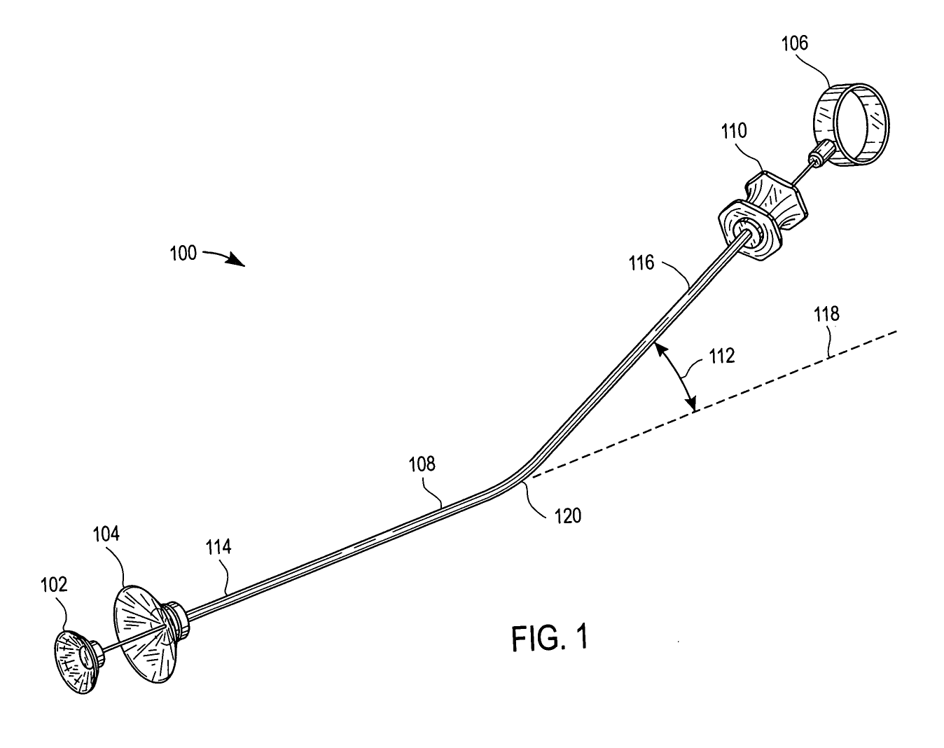

The membrane elevator in accordance with one embodiment of the present invention is shown in FIG. 1. Elongated device 100 includes a first cup 102 and a second cup 104 at one and a handle 106 at the other end. In some embodiments handle 106 is a thumb ring that is used in cooperation with finger hold 110. First cup 102 fits flush within cup 104 as illustrated. In some embodiments, cup 104 has a diameter of between 1 cm and 10 cm. In some embodiments, cup 104 has a diameter of between 1.5 cm and 9 cm. In still other embodiments, cup 104 has a diameter of between 2 cm and 8 cm. In some embodiments, cup 104 has a diameter of more than 2 cm. In still other embodiments, cup 104 has a diameter of about 8 cm or less. In some embodiments, cup 104 has a diameter of about 3.0 cm to about 5.0 cm. In some embodiments, cup 102 has any diameter so long as it can fit flush inside cup 104. In some embodiments, cup 104 has a diameter between about 1.0 cm and about 1.5 cm.

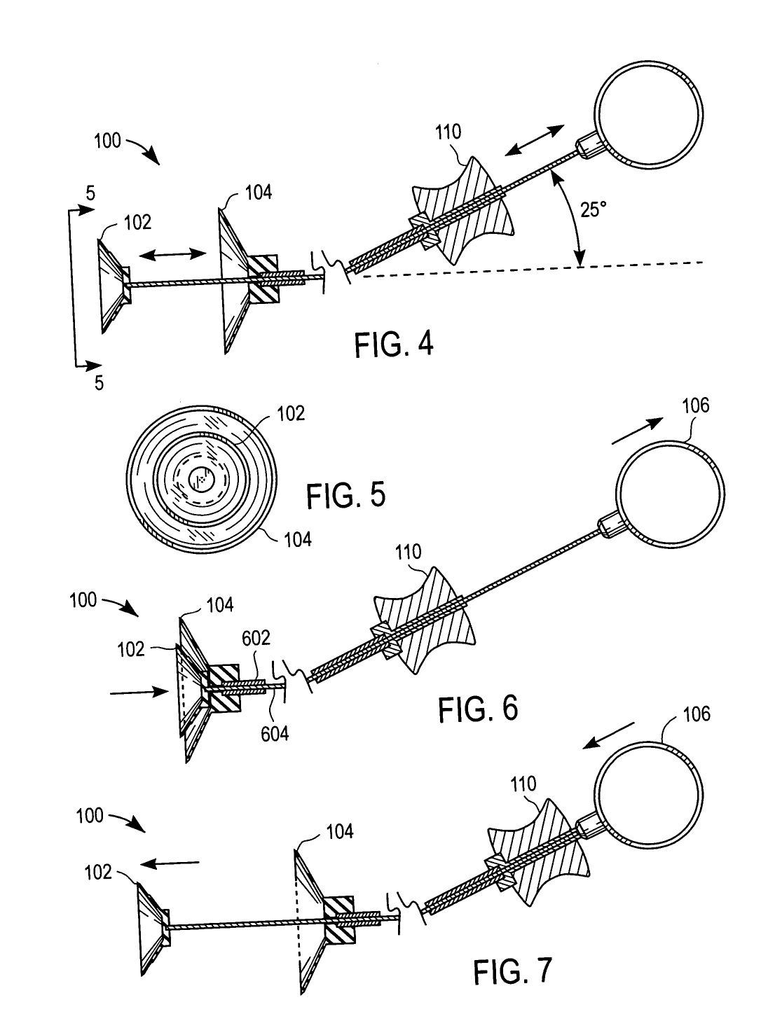

The ends of device 100 are supported by shaft 108. As illustrated, shaft 108 includes bend 112. Bend 112 is defined by the separation between (i) a portion of shaft 108 that includes a first distal end (114 or 116) and (ii) the imaginary line that is colinear with a portion of shaft 108 that includes the second distal end (116 or 114). FIG. 1 illustrates one such imaginary line, line 118. FIG. 4 illustrates another view of an imaginary line about which the angle of bend 112 is defined. In some embodiments, bend 112 ranges anywhere from between 10° and 50°. In some embodiments, bend 112 ranges anywhere from between 15° and 30°. In one example, bend 112 is about 20°. In the embodiment illustrated in FIG. 1, bend 112 is effected by a single kink 120 in shaft. However, the present invention is not limited to such embodiments. In fact, bend 112 can be effected by any number of kinks in shaft 108 (e.g., 2 kinks, 3 kinks, 4 kinks, etc.) so long as the angle between distal ends 114 and 116 falls within one of the specified ranges of the present invention. In some embodiments, there is no bend, or no appreciable bend in shaft 108. In some embodiments, shaft 108 has a minimum bend ratio of 5:1, 8:1, or 10:1.

In preferred embodiments, cup 104 and/or cup 102 are made of a soft pliable material such as a soft plastic. In some embodiments, cups 102 and 104 are made of rubber, a rubberlike material, a rubber derivative, silicone rubber, or an elastomer. In some embodiments, cup 104 and cup 102 are made of natural rubber, vulcanized rubber, a butadiene-styrene polymer such as GR-S, neoprene, nitrile rubbers, butyl, polysulfide rubber, ethylene-propylene rubber, polyurethane rubber, silicone rubber, gutta-percha, and/or balata. In some embodiments cup 104 and/or cup 102 is made of silicone rubber. Silicone rubber is a rubberlike material having a tensile strength of between 400 lb/in² to 700 lb/in² (2.78 to 4.85×10⁶ N/m²) elongation. In some embodiments, cups 102 and 104 are made of Silastic® silicone rubber (Dow Corning).

In some embodiments, cups 102 and 104 are made out of the same material. In some embodiments, cups 102 and 104 are made out of different materials. As used herein the term elastomer is used to describe both natural and synthetic materials which are elastic or resilient and in general resemble natural rubber in feeling and appearance. See, for example, Avallone and Baumeister III, Marks' Standard Handbook for Mechanical Engineers, McGraw Hill, 1987, which is hereby incorporated by reference.

In some embodiments, cups 102 and 104 are made out of a plastic or a rubber. In some embodiments, cups 102 and 104 are made out of high-density polyethylene, low-density polyethylene, polypropylene, cellulose acetate, vinyl, plasticized vinyl, cellulose acetate butyrate, melamine-formaldehyde, polyester, nylon. See, for example, Modern Plastics Encyclopedia, McGraw-Hill.

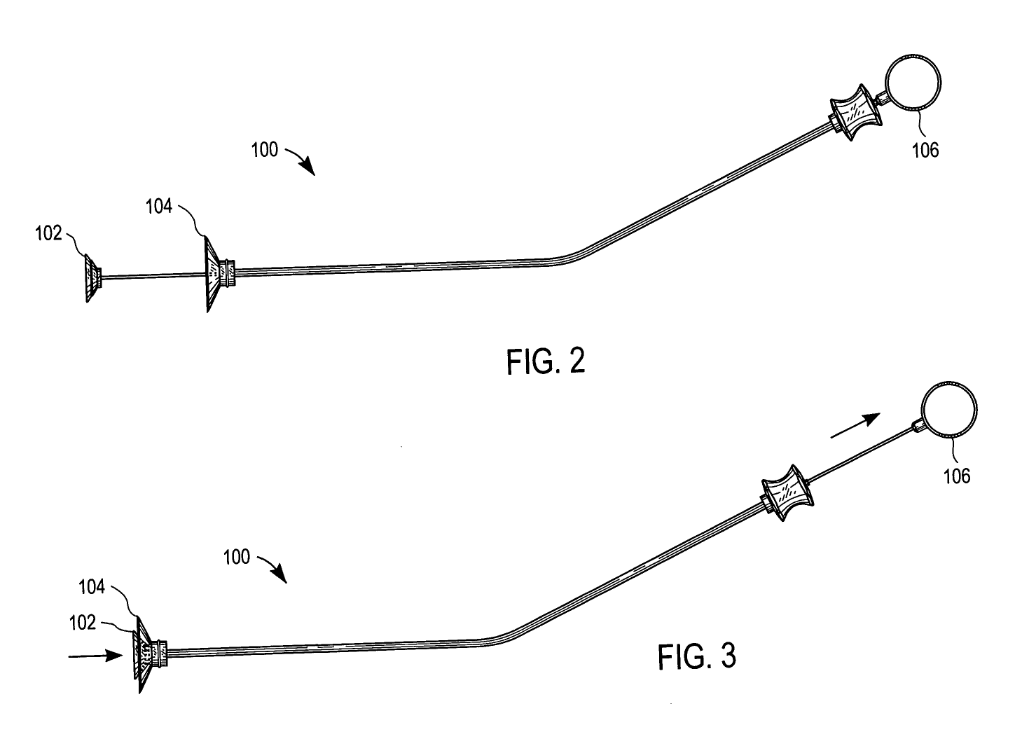

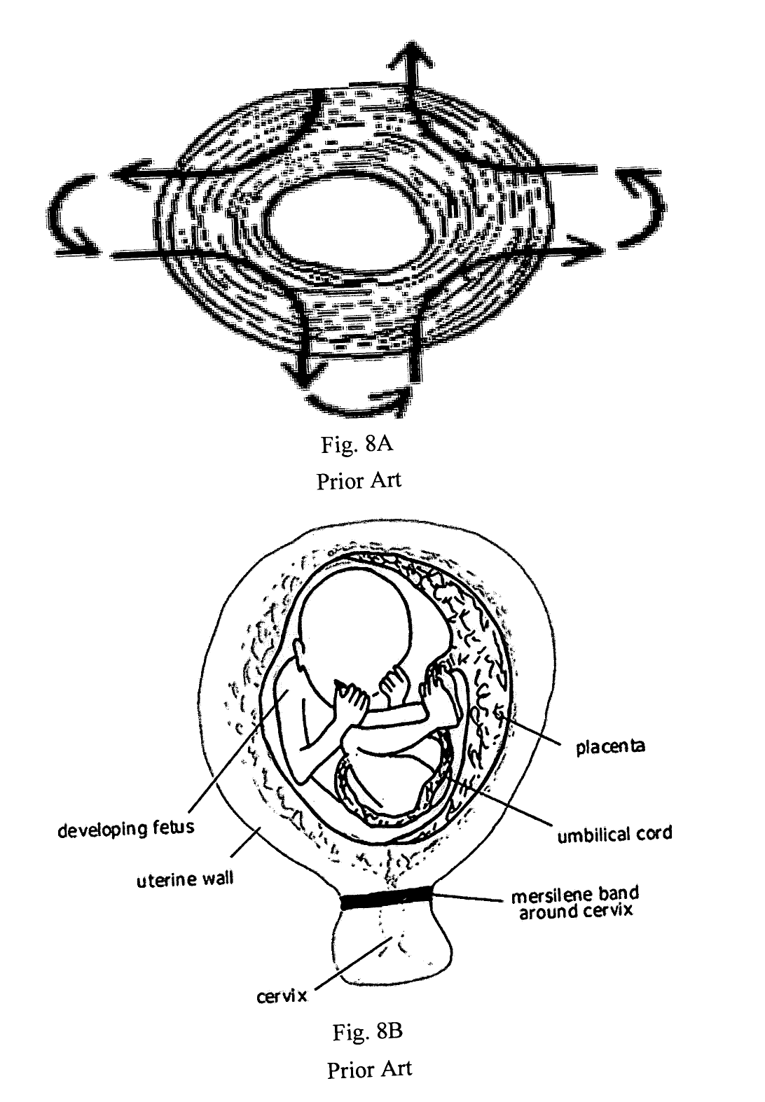

In the present invention, distal end 114 is inserted into the vagina up to the cervical os while in the closed position illustrated in FIG. 3 or in a near closed position. Cup 104 is held firmly against the bulging membrane until the cervix is covered. Then, cup 102 is deployed using handle 106. Through this operation, cup 102 is deployed to the extended position illustrated in FIG. 2 so that cup 102 gently pushes the membrane of the amniotic sac upwards and back into the cervix. Then, a purse string type cerclage, such as that illustrated in FIG. 8A, is initiated while cup 102 is deployed in the extended position illustrated in FIG. 2. Once the cerclage has been initiated, cup 102 can be removed from the cervix and the cerclage drawn tight in a single fluid motion to achieve the state shown in FIG. 8B. In some embodiments, cup 102 is removed from the cervix by withdrawing device 100 from the cervix. In some embodiments, cup 102 is removed from the cervix by returning device 100 to the closed position illustrated in FIG. 2. The fluid operation entailing removal of cup 102 from the cervix (see, for example, FIG. 8B for an illustration of a cervix) as the cerclage is tightened is facilitated by the flexible nature of cup 102. In essence, cup 102 is squeezed as the cerclage is tightened. However, because of the flexible nature of cup 102, it does not cause damage to the cervix as it is pulled out.

FIG. 5 shows a cross-sectional view of cups 104 and 102 taken from an end on view of the device illustrated in FIG. 4. The cross-sectional view of cups 104 and 102 shown in FIG. 5 exposes the interior surface of these cups. In some embodiments, at least a portion of the interior surface of cup 102 has a radius of curvature of between 0.5 cm and 1000 cm, between 1 cm and 500, between 0.75 cm and 100 cm, between 1 cm and 10 cm, greater than 0.5 cm or less than 100 cm. In some embodiments, at least a portion of the interior surface of cup 104 has a radius of curvature of between 0.5 cm and 1000 cm, between 1 cm and 500, between 0.75 cm and 100 cm, between 1 cm and 10 cm, greater than 0.5 cm or less than 100 cm. In some embodiments, cups 104 and 102 are characterized as having the same radius of curvature. In other words, in some embodiments, a substantial portion of the interior surfaces of both cup 102 and 104 have the same radius of curvature. However, the present invention is not limited to such embodiments. In fact, in some embodiments, a substantial portion of the interior surface of cup 102 is characterized by a radius of curvature that is either less than or greater than a radius of curvature that characterizes a substantial portion of the interior surface of cup 104.

Returning to FIG. 1, in some embodiments, the overall length of device 100 is between 20 cm and 40 cm. In more preferred embodiments, the overall length of device 100 is between 22 cm and 36 cm. In still more preferred embodiments, the overall length of device 100 is between 14 cm and 31 cm. In some embodiments, the overall length of device 100 is greater than 15 cm. In some embodiments, the overall length of device 100 is less than 40 cm.

FIG. 6 is a cross-sectional view of device 100 drawn about line 5-5 of FIG. 4. As illustrated in FIG. 6, device 100 includes a primary tube 602 and an interior member 604. Cup 104 and finger hold are fixably mounted on primary tube 602 whereas cup 102 and handle 106 are fixably mounted on interior member 604. In preferred embodiments, primary tube 602 is made of a rigid or semi-rigid component that is bendable. In preferred embodiments, primary tube 602 includes bend 120 whereas member 604 is made out of a substantially flexible material that flexibly fits inside primary tube in a slideable fashion. In some embodiments, primary tube 602 and/or interior member 604 is made out of any of the materials that can be used to make cups 102 and 104. In some embodiments, primary tube 602, interior member 604, cups 102 and 104 are made out of the same or different materials.

FIG. 6 illustrates device 100 when it is in a closed position. In such embodiments, member 604 protrudes past finger hold 110 and/or primary tube 602 by a distance of between 3 cm and 20 cm, between 4 cm and 17 cm, between 5 cm and 10 cm, between 3 cm and 5 cm, more than 2 cm and/or less than 10 cm when device 100 is fully in the closed position as illustrated in FIG. 6. Then, to elevate cup 102, handle is pushed in the direction illustrated by the arrow in FIG. 7. This pushing operation causes interior member 604 to slide freely through primary tube 602, thereby causing cup 102 to be displaced from the interior or near interior of cup 104. Once handle has been pushed such that it contacts finger hold 110, device 100 is fully in the elevated (open) position and cup 102 has been fully displaced away from cup 104 in the manner illustrated in FIG. 7. As such, in the fully extended position illustrated in FIG. 7, cup 102 and cup 104 are displaced by a distance of between 3 cm and 20 cm, between 4 cm and 17 cm, between 5cm and 10 cm, between 3 cm and 5 cm, more than 2 cm and/or less than 10 cm when device 100 is fully in the extended (open) position illustrated in FIG. 7.

In preferred embodiments of the present invention, one or more of the components of device 100 are made of a plastic or a rubber, such as any of the materials described in detail with respect to cup 102 and 104. Such embodiments are particularly advantageous because the cost of manufacture of such devices is relatively inexpensive. Thus, a preferred embodiment of the present invention are devices 100 that are disposable and/or are made of disposable materials. In another preferred embodiment of the present invention, one or more components of device 100 are made of a material that is biodegradable or is otherwise recycleable.

In another method in accordance with the present invention, distal end 114 is inserted into the vagina up to the cervical os while in the closed position illustrated in FIG. 3 and FIG. 6 or in a near closed position. Cup 104 is held firmly against the bulging membrane until the cervix is covered. Then, cup 102 is deployed using handle 106. Through this operation, cup 102 is deployed to the extended position illustrated in FIG. 2 so that cup 102 gently pushes the membrane of the amniotic sac upwards and back into the cervix. Then, finger hold 110 is removed or withdrawn. In some embodiments, finger hold 110 is removed or withdrawn by any of a number of means including, but not limited to, unscrewing finger hold 110 from primary tube 602, releasing finger hold 110 from primary tube 602 by releasing a pinch clip, or other means. Removal of finger hold 110 allows for the removal of primary tube 602 and cup 104. Cup 102 is remains to support the membrane. Removal of primary tube 602 and cup 104 is advantageous because it provides better visualization during placement of the cerclage. Once the cerclage has been initiated and as it is tightened to complete the cerclage, cup 102 can be simultaneously pulled out of the cervix thereby completing the operation.

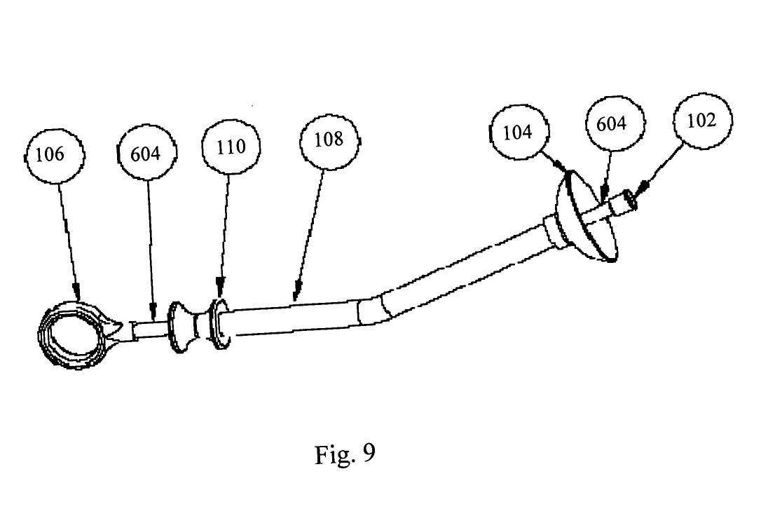

FIG. 9 illustrates another embodiment of the present invention in which cup 104 has an inner surface with a substantially different curvature than the inner surface of cup 102.

While examples of the use of the devices of the present invention have been given with reference to humans, the device can be used with other species as well. In particular, it is known that horses can be prone to premature dilation of the cervix. This is a particularly important issue in thoroughbred breeding, wherein each newborn horse has the potential to be awarded millions of dollars in revenue. It will be appreciated that the dimensions of cup 102 and 104 and the overall dimensions of the apparatus would need to be dimensioned for each type of animal that for which use is contemplated. In some embodiments, the apparatus of the invention is used for horses, cows, dogs, elephants, lions, and other mammals.

All references cited herein are incorporated herein by reference in their entirety and for all purposes to the same extent as if each individual publication or patent or patent application was specifically and individually indicated to be incorporated by reference in its entirety for all purposes.

The foregoing descriptions of specific embodiments of the present invention are presented for purposes of illustration and description. They are not intended to be exhaustive or to limit the invention to the precise forms disclosed. Obviously many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents.

1. A device comprising:
a primary tube;
an interior member slideably mounted within said primary tube, wherein said interior member has a length that is longer than a length of said primary tube and said interior member protrudes from a first and second end of said primary tube;
a first cup fixably mounted on said first end of said primary tube, said first cup having an opening through which said interior member protrudes;
a finger hold fixably mounted on said second end of said primary tube;
a second cup fixably mounted on a first end of said interior member wherein all or a portion of said first end of said interior member protrudes through said first cup; and
a handle fixably mounted on a second end of said interior member, wherein all or a portion of said second end of said interior member protrudes from said second end of said primary tube.

2. The device of claim 1 wherein said handle is a thumb ring.

3. The device of claim 1 wherein said first cup has a diameter between about 2 cm and 8 cm.

4. The device of claim 1 wherein said first cup has a diameter between about 3.0 cm and 5.0 cm.

5. The device of claim 1 wherein said second cup is dimensioned and configured to fit flush within said first cup.

6. The device of claim 1 wherein said second cup has a diameter between about 1.0 cm and about 1.5 cm.

7. The device of claim 1 wherein said primary tube has a bend.

8. The device of claim 7 wherein said bend ranges between 10° and 50°.

9. The device of claim 7 wherein said bend ranges between 10° and 50°.

10. The device of claim 1 wherein said primary tube has a minimum bend ratio of 5:1.

11. The device of claim 1 wherein said first cup or said second cup are made of a rubber, a rubberlike material, a rubber derivative, or silicone rubber.

12. The device of claim 1 wherein said first cup or said second cup are made of an elastomer.

13. The device of claim 1 wherein said first cup is made out of a first material and said second cup is made out of a second material.

14. The device of claim 1 wherein said first cup and said second cup are made out of the same material.

15. The device of claim 1 wherein said first cup has an interior surface that faces away from said primary tube, and wherein all or a portion of said interior surface is characterized by a radius of curvature.

16. The device of claim 15 wherein said radius of curvature is between 0.5 cm and 1000 cm.

17. The device of claim 15 wherein said radius of curvature is between 1 cm and 10 cm.

18. The device of claim 15 wherein said radius of curvature is less than 100 cm.

19. The device of claim 1 wherein said second cup has an interior surface that faces away from said interior member, and wherein all or a portion of said interior surface is characterized by a radius of curvature.

20. The device of claim 19 wherein said radius of curvature is between 0.5 cm and 1000 cm.

21. The device of claim 19 wherein said radius of curvature is between 1 cm and 10 cm.

22. The device of claim 19 wherein said radius of curvature is less than 100 cm.

23. The device of claim 1 wherein an overall length of said device is between 20 cm and 40 cm.

24. The device of claim 1 wherein said primary tube is made out of a rubber, a rubberlike material, a rubber derivative, or silicone rubber.

25. The device of claim 1 wherein said first device is characterized by a closed position, wherein, when said device is in said closed position, said second end of said interior member protrudes past said second end of said primary tube by a first distance.

26. The device of claim 25 wherein said first distance is between 3 cm and 20 cm.

27. The device of claim 25 wherein said first distance is between 3 cm and 5 cm.

28. The device of claim 25 wherein said device is further characterized by an open position, wherein, when said device is in said open position, said first cup and said second cup are displaced by a second distance/

29. The device of claim 28 wherein said second distance is between 3 cm and 20 cm.

30. The device of claim 28 wherein said second distance is between 3 cm and 5 cm.

31. The device of claim 28 wherein said device is disposable.

32. The device of claim 28 wherein said device is recycleable.

33. A method of implementing a cerclage, the method comprising:
(A) inserting a distal end of a device into the vagina up to the cervical os while said device is in a closed position, wherein a second cup of said device is located circumferentially within a first cup of said device, until said first cup is pressing against a uterine wall;
(B) deploying said second cup into the cervix of the uterus thereby causing said second cup to be deployed in an extended position that pushes a membrane of the amniotic sac into the cervix;
(C) initiating a cerclage of the cervix; and
(D) withdrawing said second cup from said cervix as the cerclage is tightened, thereby implementing said cerclage.

34. The method of claim 33 wherein said cerclage is in accordance with the McDonald approach.

35. The method of claim 3 wherein after said step (B) and prior to said step (C) said first cup is removed from said vagina.
